(19) United States
(12) Patent Application Publication
(10) Pub. No.: US 2025/0381351 A1
(43) Pub. Date: Dec. 18, 2025

(54) COLLAPSIBLE ASTHMA SPACER

(71) Applicant: AERAFLOW, LLC, Provo, UT (US)

(72) Inventors: Brian James HALL, Provo, UT (US); Matthew Ray PETERSEN, Provo, UT (US)

(21) Appl. No.: 18/877,168

(22) PCT Filed: Feb. 28, 2024
(86) PCT No.: PCT/US24/17734

§ 371 Date: Dec. 19, 2024

Related U.S. Application Data
(60) Provisional application No. 63/448,867, filed on Feb. 28, 2023.

(51) Int. Cl. A61M 15/00

(52) U.S. Cl. CPC A61M 15/0086; A61M 2205/02

(57) ABSTRACT

A collapsible spacer for use with an inhaler or similar device can include a main body including a flexible material having antistatic properties. The main body may be adjusted between a collapsed or nested arrangement and an extended arrangement. The main body may define an interior volume therein for receiving an airborne medication. An outlet component may be provided at a first distal end of the collapsible spacer for a user to inhale the medication therethrough. A receiver component may be provided at a second distal end of the collapsible spacer for receiving the inhaler or similar device and for dispensing the medication therethrough.

### TECHNICAL FIELD

The disclosure relates to inhalation chambers and spacer devices for facilitating delivery of medication and related therapeutics for inhalation by a user. The devices of the disclosure may be used with related medical devices for delivering medicines into the lungs, such as an inhaler.

### BACKGROUND

Medical devices, such as inhalers, are commonly used to deliver medication into a user's lungs by the respiratory action of the user, i.e., inhaled through the mouth and into the lungs. Inhalers can be used to treat medical conditions such as asthma, chronic obstructive pulmonary disease (COPD), emphysema, chronic bronchitis, etc., and have been successfully used for many years. However, despite their generally accepted convenience, known inhaler devices can be limited in efficacy by the skill and understanding of the user. This limited efficacy can be attributed to the need for a coordinated and slow delivery of the medication from the inhaler into the airways of the user, which is frustrated by the velocity at which the medication is released from the inhaler and the need for the user to coordinate activation of the inhaler with slow, deep inhalation.

One solution for improving the efficacy of an inhaler device relies on the use of a spacer device, in the form of a chamber or reservoir placed between the inhaler device and the user. The use of a spacer device slows down the medication released by the inhaler device, allowing the user more time for slow, coordinated breathing and reducing the amount of medication that impacts and is deposited in the mouth and throat.

Despite the well-known and established benefits of spacer devices, they remain widely underused. This may be attributed to the fact that known spacer devices are bulky and rigid, making them inconvenient and uncomfortable to transport. This inconvenience is particularly problematic due to the benefit of a spacer device being most pronounced during emergency administration, which is most often needed when the user is away from home and in unfamiliar or outdoor environments.

While many attempts have been made at forming more convenient spacer devices, known designs remain bulky and rigid. Further, other designs have sacrificed other important efficiencies for convenience, leaving users in need of a more convenient and efficient spacer device.

Accordingly, there remains a need for a spacer device that is convenient to transport while remaining capable of delivering a maximally effective dose of medication to a user.

### SUMMARY

Embodiments of the present disclosure advantageously provide a collapsible spacer in the form of a main body defining an interior volume within, the main body formed of a flexible material having antistatic properties. The main body may be configured to move between a collapsed configuration and an extended configuration, such that the extended configuration provides the interior volume for dispersion of medication. An outlet component may be provided at a first distal end of the collapsible spacer and a receiver component may be provided at a second distal end of the collapsible spacer, wherein the receiver component is configured to allow medication to enter the interior volume of the main body.

According to an embodiment, the flexible material of the main body of the collapsible spacer may comprise one or more of silicone, rubber, thermoplastic polyurethane (TPU), thermoplastic elastomers (TPE) and/or other flexible materials. The material of the collapsible spacer may comprise an antistatic material and/or include an antistatic additive, such that the material provides antistatic properties. An antistatic additive may comprise carbon black, graphene nanotubes, conductive mica, and/or carbon fiber, or the like, and may be mixed and/or fully integrated in the polymer or elastomer forming the flexible material.

The main body of the collapsible spacer may have a substantially conical or frustoconical shape and may be configured to collapse or fold along folding portions defined therein from an extended configuration into a collapsed or nested configuration. A narrowest portion may be defined in the main body with a smallest diameter, the narrowest portion advantageously allowing for an improved transition between an extended configuration and a collapsed configuration. A diameter of the main body in the extended configuration may decrease monotonically from the second distal end to the narrowest portion of the main body, and the diameter of the main body may increase from the narrowest portion to the first distal end. Advantageously, a diameter of the main body may have a percent reduction in a range of 72% to 78% from the second distal end to the first distal end.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not, therefore, to be considered to be limiting in scope, embodiments will be described and explained with additional specificity and details through the use of the accompanying drawings in which:

FIG. 1 is a perspective view of a collapsible spacer in an extended configuration according to an embodiment of the disclosure.

FIG. 2 is a perspective view of a cross-section of a collapsible spacer in an extended configuration according to an embodiment of the disclosure.

FIG. 3A is a plan view of a collapsible spacer in an extended configuration and without a receiver component according to an embodiment of the disclosure.

FIG. 3B is a plan view of a collapsible spacer in an extended configuration and with a receiver component according to an embodiment of the disclosure.

FIG. 3C is a transparent plan view of a collapsible spacer in an extended configuration and without a receiver component according to an embodiment of the disclosure.

FIG. 4 is a top view of a collapsible spacer in an extended configuration according to an embodiment of the disclosure.

FIG. 5 is a bottom view of a collapsible spacer in an extended configuration according to an embodiment of the disclosure.

FIG. 6 is a perspective view of a collapsible spacer in a collapsed configuration according to an embodiment of the disclosure.

FIG. 7 is a perspective view of a collapsible spacer in an intermediate configuration according to an embodiment of the disclosure.

FIG. 8 is a perspective view of a collapsible spacer in an intermediate configuration according to an embodiment of the disclosure.

FIG. 9 is a perspective view of a collapsible spacer in an intermediate configuration according to an embodiment of the disclosure.

FIG. 10 is a perspective view of a collapsible spacer in an extended configuration according to an embodiment of the disclosure.

FIG. 11 is a perspective view of a cross-section of a collapsible spacer in a collapsed configuration according to an embodiment of the disclosure.

The drawing figures are not necessarily drawn to scale, but instead are drawn to provide a better understanding of the components, and are not intended to be limiting in scope, but to provide exemplary illustrations. The figures illustrate exemplary configurations of collapsible spacers, and in no way limit the structures or configurations of spacer devices according to the present disclosure.

### DESCRIPTION

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. It should be understood, however, that there is no intention to limit the disclosure to the specific embodiments disclosed, but on the contrary, the intention covers all modifications, alternative constructions, combinations, and equivalents falling within the spirit and scope of the disclosure. The dimensions, angles, and curvatures represented in the figures introduced above are to be understood as exemplary and are not necessarily shown in proportion. The embodiments of the disclosure may be adapted or dimensioned to accommodate use with different inhalers, mouthpieces, respirators, etc., as would be understood from the present disclosure by one skilled in the art.

Certain embodiments and features may be described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Any numerical value is “about” or “approximately” the indicated value, and takes into account experimental error and variations that would be expected by a person having ordinary skill in the art.

It will be understood that unless a term is expressly defined in this application to possess a described meaning, there is no intent to limit the meaning of such term, either expressly or indirectly, beyond its plain or ordinary meaning. Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

The terms “connected” and “secured,” and variations thereof, as used herein, mean that one or more members are associated or coupled to each other, either directly or indirectly (for example with intervening members).

It is to be noticed that the term “comprising,” which is synonymous with “including,” “containing,” “having” or “characterized by,” should not be interpreted as being restricted to the means listed thereafter; it does not exclude other or additional, unrecited elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present disclosure, the relevant components of the device are A and B.

It will be noted that, as used in this specification and the appended claims, the singular forms “a,” “an” and “the” include plural referents unless the content clearly dictates otherwise.

Reference throughout this specification to “one embodiment,” “one aspect,” or “an embodiment” means that a particular feature, structure or characteristic described in connection with the embodiment is included in at least one embodiment of the present disclosure. As used herein, the term “embodiment” or “aspect” means “serving as an example, instance, or illustration,” and should not necessarily be construed as preferred or advantageous over other embodiments disclosed herein. Thus, appearances of the phrases “in one embodiment,” “in one aspect,” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the embodiments require more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some, but not other features included in other embodiments, combinations of features of different embodiments are meant to be within the scope of the disclosure, and form different embodiments, as would be understood by those in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

The various embodiments of the disclosure relate to a collapsible spacer (e.g., collapsible asthma spacer). The collapsible spacer may be used with a medical device, such as an inhaler, for the delivery of medication to a user. An inhaler (e.g., metered dose inhalers, dry powder inhalers, pressurized metered dose inhaler (pMDI), or the like) may include a pressurized canister of medicine—which releases a fine mist or powder when depressed—and a mouthpiece. When using an inhaler without a spacer device, a user may place the mouthpiece in their mouth, depress the pressurized cannister to release the medicine, and inhale deeply as the medicine is dispersed.

Although certain embodiments of the present disclosure refer to asthma or a collapsible asthma spacer, embodiments of the present disclosure may be used for respiratory conditions other than asthma. Similarly, it should be understood that a collapsible spacer can also be used in conjunction with medication delivery containers other than an inhaler, for example including ventilators, nasal sprayers, dry powder inhalers, nebulizer systems, and the like.

Unfortunately, when medicine is administered with just a conventional inhaler, most of the medicine gets stuck in the mouth and throat and never reaches the lungs where it needs to go. Even with experienced technique, it is difficult for a user to get more than 20% of an emitted dose into the lower airways using just an inhaler. If the user incorrectly coordinates their breath with the medicine dispersal, coughs when the medicine hits their mouth, and/or improperly angles their airway to the inhaler, the user will receive significantly less, if any, medication.

To address these challenges, a spacer may be prescribed for use in conjunction with the inhaler when taking medicine. A spacer may be a device that has a receiver piece at a first distal end (for the inhaler or medicine canister to be inserted or connected), a holding chamber at the center, and an outlet at a second distal end. Conventional spacers are commonly made of hard plastic and can look like a tube that has the receiver at the first distal end and the outlet at the second distal end.

When taking medication using an inhaler with a spacer device, the user inserts the inhaler or medicine canister into the receiver of the spacer and places their mouth (or attaches a face mask or the like) on the outlet. When the medicine is released, it enters the holding chamber and slows down. The user can then inhale the medicine when ready, and the medicine can be inhaled into the lungs without the majority of the medicine being deposited in the mouth or the throat. Using an asthma spacer with an inhaler enhances the delivery of medication and is especially helpful for children, the elderly, or anyone who has difficulty following the relatively complex steps of using an inhaler correctly. Spacers are likewise important for users that have severe asthma, as these users require that a higher dose of medication is dispersed throughout the entirety of their lungs. Without a spacer, a user may not receive enough medication in the event of a severe asthma attack.

A conventional spacer, which is about the size of an average water bottle, is typically too large to be easily portable, and is very awkward and inconvenient to carry around. As a result, many users with asthma or other respiratory illnesses, including those who suffer from a severe diagnosis or frequent attacks, will often take a risk and opt to not carry their spacer around with them. The result is that, in the event of an attack, these users usually must take their medication three to six times to receive an adequate dose. For those with a severe diagnosis or frequent attacks, even this is often not enough due to the dispensed doses failing to reach the lungs. About one quarter of all emergency room visits each year in the United States are related to asthma and there are over 4,000 asthma related deaths in the United States each year—most of these could have been prevented with the proper use of a spacer.

Embodiments of a collapsible spacer according to the present disclosure may include a main body, an outlet component, and a receiver component. The main body may include a flexible material having antistatic properties which prevent an airborne medication from sticking to the main body. The main body may be configurable between a collapsed configuration and an extended configuration. The main body in the extended configuration may define an interior volume for dispersion of medication (e.g., albuterol, epinephrine, levalbuteral, etc.). The outlet component may be disposed at a first distal end of the collapsible spacer and may be configured to couple to a mouth of a user, a mouthpiece, a mask, or the like for inhalation by the user. The receiver component may disposed at a second distal end of the collapsible spacer opposite the first distal end. The receiver component may be configured to attach to an inhaler, such as a metered-dose inhaler or the like.

Embodiments of a collapsible spacer according to the present disclosure may advantageously increase the effectiveness, efficiency and convenience of administering medication for respiratory diseases. Advantageously, the disclosed embodiments of collapsible spacers may be made from flexible materials having antistatic properties, allowing the spacer to readily and easily move between an extended and a collapsed configuration without limiting an effective dose available to the user. In varying embodiments, a collapsible spacer may include an outlet component for dispersing the medication to a user, a receiver component for connecting an inhaler canister or medicine container, and a main body defining an interior volume (e.g., main holding chamber). The collapsible spacer may be configured to collapse into a closed or collapsed configuration for easy and convenient transport. The outlet component and receiver component may be separate or continuous with the main body. The collapsible spacer may be made of one or more of silicone, rubber, thermoplastic polyurethane (TPU), thermoplastic elastomers (TPE) and/or other flexible materials. The material of the collapsible spacer may comprise an antistatic material and/or include an antistatic additive, such that the material provides antistatic properties.

Although certain embodiments of the present disclosure refer to asthma or a collapsible asthma spacer, embodiments of the present disclosure may be used for respiratory conditions other than asthma.

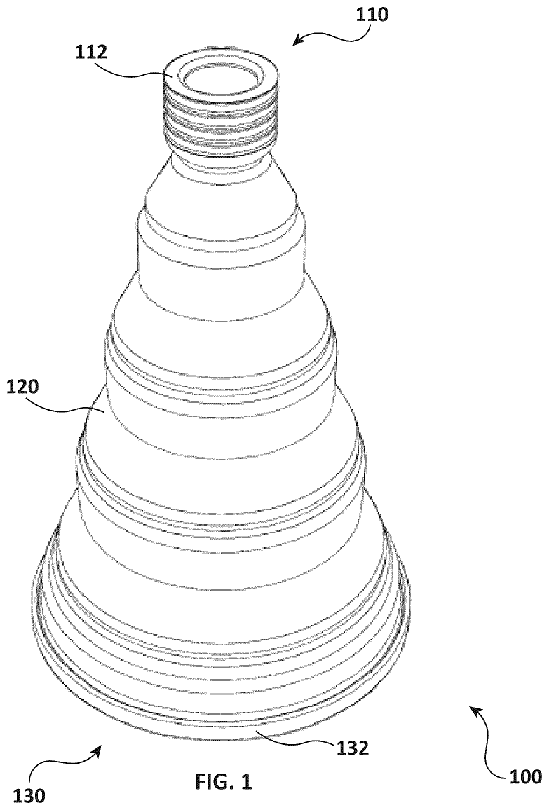

Depicted in FIG. 1 is a perspective view of an embodiment of a collapsible spacer 100 incorporating features of the present disclosure. As seen in an extended configuration according to FIG. 1, the collapsible spacer 100 may comprise a first distal end 110 defining an outlet component 112, a main body 120 defining an interior volume therein, and a second distal end 130 defining a receiver component 132. In use, the collapsible spacer 100 may be configured to receive an inhaler or related device for dispensing medication at the receiver component 132, such that medication may be introduced into the interior volume of the main body 120 before the medication passes through the outlet component 112, for example by a user inhaling the medication through the outlet component 112.

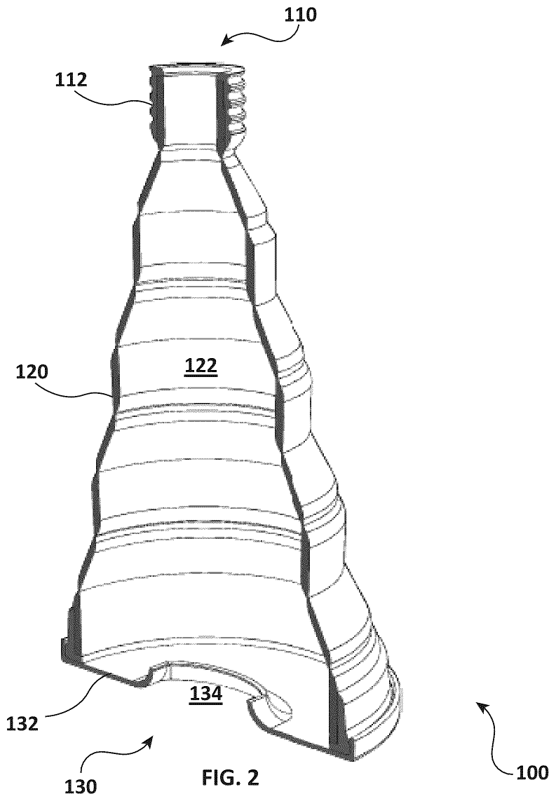

As seen in the illustrated perspective view of a cross-section of an embodiment of a collapsible spacer according to FIG. 2, the interior volume 122 of the depicted embodiment may be dimensioned and configured to receive and allow for the deceleration of an administered dose of medication. Notably, metered-dose inhalers and related devices may dispense pressurized doses of medication, such as a powdered medication, at velocities of as high as 60 miles per hour. At high velocities, the medication may impact and stick to encountered surfaces, such as in the mouth and throat, rather than pass to the lungs where the medication is needed. As such, embodiments of the current disclosure may provide an interior volume 122 sufficiently dimensioned so as to reduce a velocity of the medication and allow a user to inhale the medication in a coordinated manner.

According to the embodiments of FIG. 1 and FIG. 2, the main body 120 may have a substantially conical or frusto-conical shape. Notably, the substantially conical or frusto-conical shape of the depicted embodiment differs from the generally cylindrical shapes used in conventional spacer devices. Conventional spacer devices have been designed with substantially cylindrical shapes in an effort to conform to a shape of a plume of medication formed by inhaler devices when dispensing medication. The conventional belief appears to be that the substantially cylindrical shape allows a sufficient volume for slowing the dispensed plume while avoiding the medication impacting the walls of the spacer. Surprisingly, the conical or frustoconical shape of the current disclosure has been found to improve medication delivery while allowing for a collapsible nesting configuration that significantly reduces the inconvenience in transporting the spacer relative to cylindrical spacer devices. In particular, the conical shape allows a center of a plume of medication to approach an outlet directly, while redirecting an expanding edge of the plume towards the outlet.

In the embodiments of FIG. 1 and FIG. 2, the outlet component 112 is located at the first distal end 110, the first distal end 110 having a smaller diameter than the second distal end 130 where the receiver component 132 is located. In this way, an inhaler may be introduced to the spacer at the second distal end 130, such as in a cavity 134 defined by the receiver component 132 and dimensioned to connect with the inhaler, or in other words at an area of the conical or frustoconical shape having a larger volume than at the outlet component 112. This arrangement advantageously allows for a channeling effect where the medication dispersed into the interior volume 122 of the spacer 100 is concentrated towards the outlet component 112 upon inhalation by a user, increasing an amount of medication delivered from the interior volume 122 of the spacer 100 to the user in addition to allowing the medication to decelerate before inhalation.

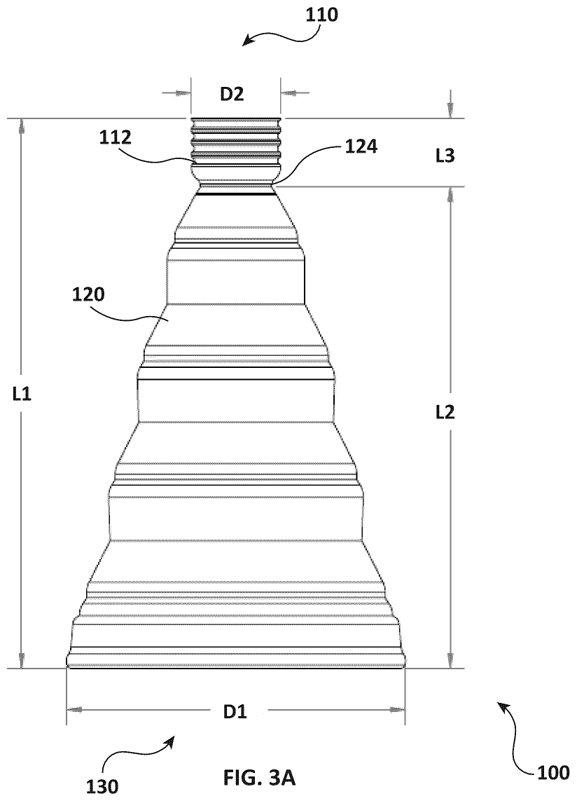

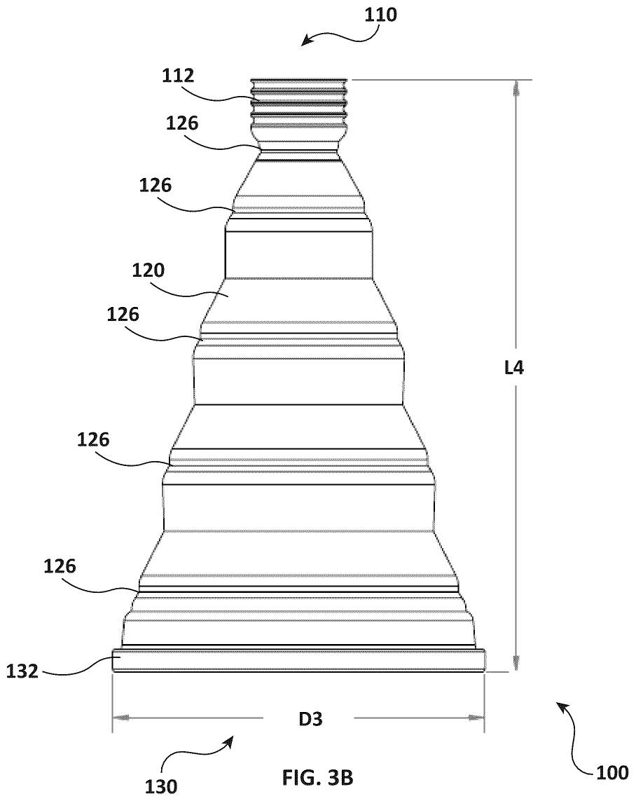

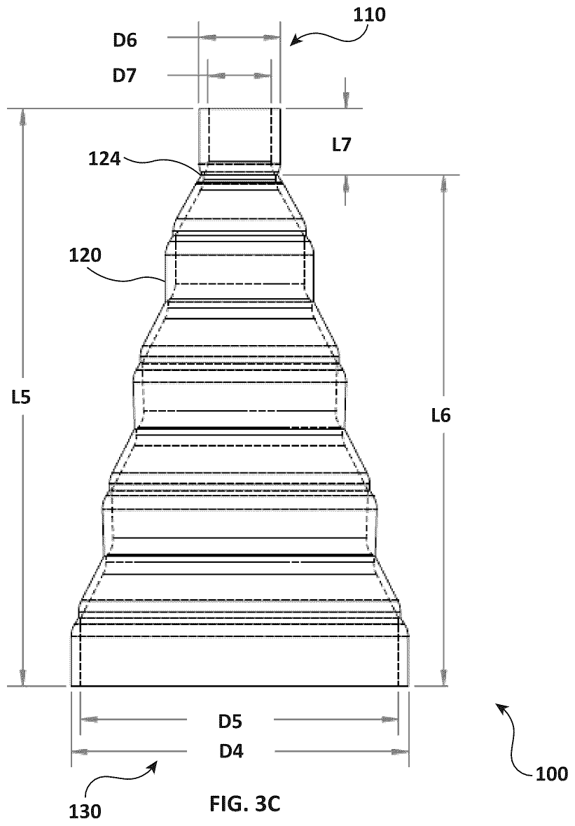

As shown in the plan views of FIGS. 3A to 3C, according to varying embodiments, the main body 120 of the collapsible spacer 100 may have diameter reduction in a range of 51% to 85%, particularly in a range of 65% to 80%, more particularly in a range of 72% to 78% from the second distal end 130 to the first distal end 110. As discussed above, this reduction in diameter advantageously channels airborne medication into the airways of a user upon inhalation. In certain embodiments, a diameter of the first distal end may be in a range of 1.25 to 2 cm. A diameter of the second distal end may be in a range of 6.5 to 8.0 cm. A length of the collapsible spacer, from the first distal end to the second distal end, may be in a range of 10 to 13 cm in an extended configuration.

FIG. 3A provides a plan view of the dimensions of a collapsible spacer 100 in an extended configuration without the receiver component attached to the main body 120. In this embodiment, a length L1 from the second distal end 130 of the main body 120 to the first distal end 110 of the outlet component 112 may be in a range of 11.25 to 13.75 cm, particularly 12 to 13 cm, more particularly about 123 mm. A length L2 from the second distal end 130 of the collapsible spacer 100 to a narrowest portion 124 of the main body 120 may be in a range of 10 to 11.5 cm, particularly 10.5 to 11 cm, more particularly about 107.73 mm. The narrowest portion 124 of the main body 120 may comprise the region of the main body 120 where an external diameter of the main body 120 is at its smallest. A length L3 from the first distal end 110 of the main body 120 to the narrowest portion 124 of the main body 120 may be in a range of 1 to 2 cm, particularly 1.25 to 1.75 cm, more particularly about 15.28 mm. A diameter D1 of the main body 120 at the second distal end 130 may be in a range of 6 to 9 cm, particularly 7 to 8 cm, more particularly about 75.84 mm. A diameter D2 of the outlet component 112 at the first distal end 130 may be in a range of 1 to 3 cm, particularly 1.5 to 2.5 cm, more particularly about 20 mm.

FIG. 3B provides a plan view of the dimensions of a collapsible spacer 100 in an extended configuration with the receiver component attached to the main body 120. In this embodiment, a length L4 from the second distal end 130 of the collapsible spacer 100 to the first distal end 110 of the collapsible spacer 100 may be in a range of 11.5 to 13.5 cm, particularly 12 to 13 cm, more particularly about 124.02 mm. The length L4 may be greater than or about equal to the length L1. A diameter D3 of the receiver component 132 at the second distal end 130 may be in a range of 6 to 9 cm, particularly 7 to 8 cm, more particularly about 77.9 mm. The diameter D3 may be greater than or about equal to the diameter D1.

As seen in the depicted embodiments, the main body 120 of the collapsible spacer 100 may be configured to have a decreasing diameter from the second distal end 130 to the first distal end 110. In some embodiments, the diameter of the main body 120 may decrease monotonically from the second distal end 130 to a narrowest portion 124 of the main body 120 before increasing towards the first distal end 110, improving a nesting ability of the collapsible spacer 100.

Advantageously, embodiments of the current disclosure have shown that providing a narrowest portion 124 of the main body 120, before an increase in diameter towards the first distal end 110, makes collapsing the main body 120 surprisingly easy. In particular, the arrangement of the narrowest portion 124 enables a rapid initiation of the folding of a plurality of folding portions 126 defined in the main body 120, such that the main body 120 readily folds into a collapsed configuration when pressure is applied from the first distal end 110 to the second distal end 130. Advantageously, the narrowest portion 124 is provided as the fold of the plurality of folds that is closest to the first distal end 110. Absent this configuration, a collapsible main body comprising a flexible material has a tendency to squish or compress rather than fold in a configured pattern. As such, this improvement realizes a significant increase in convenience of use, making a user far more likely to bring and use the collapsible spacer for administering airborne medications.

It will be appreciated that the main body 120 may be made of or otherwise include one or more flexible materials such as silicone, rubber, thermoplastic polyurethane (TPU), thermoplastic elastomers (TPE) and/or other flexible materials. The material of the collapsible spacer may comprise an antistatic material and/or include an antistatic additive, such that the material provides antistatic properties. An antistatic additive may comprise carbon black, graphene nanotubes, conductive mica, and/or carbon fiber, or the like, and may be mixed and/or fully integrated in the polymer or elastomer forming the flexible material. In some embodiments, the flexible material may have a surface resistivity of between about 10E10 and about 10E12 ohm/sq, or a surface resistivity of less than about 10E12 ohm/sq.

According to varying embodiments, the flexible material of the main body 120 may have a shore A hardness of 25 to 80, particularly of 35 to 65, or more particularly of 50 to 60. The flexibility of the described materials provides the significant advantage of a collapsible main body, configurable between an expanded configuration to be used for administering medication from an inhaler and a collapsed configuration to be used in transport and storage of the spacer. In preferred embodiments, the main body 120 main comprise a continuous element of flexible material, such as may be formed by a molding process, e.g., injection molding. Such a continuous material provides significant advantages over a multi-piece arrangement in effectiveness, ease of use, reduction of static electricity generation, and for cleaning.

In certain embodiments, a wall thickness of the main body may vary across the length of the main body from the first distal end to the second distal end. FIG. 3C provides a transparent plan view of a main body 120 of a collapsible spacer 100 without the outlet component 112 and the receiver component 132 attached, where a varying thickness of the main body 120 is depicted. For example, the main body may have a wall thickness in a range of 0.25 to 0.5 cm, particularly 0.3 to 0.45 cm, allowing the main body sufficient resiliency to be stable in either the expanded and collapsed configurations while also being flexible enough to fold or otherwise transition from one of the expanded and collapsed configurations to the other.

According to the embodiment of FIG. 3C, a length L5 from the second distal end 130 of the main body 120 to the first distal end 110 of the main body 120 may be in a range of 11.25 to 13.75 cm, particularly 11.75 to 13.25 cm, more particularly about 120 mm. A length L6 from the second distal end 130 of the main body 120 to a narrowest point 124 of the main body 120 may be in a range of 10 to 11.5 cm, particularly 10.5 to 11 cm, more particularly about 106.22 mm. A length L7 from the first distal end 110 of the main body 120 to the narrowest point 124 of the main body 120 may be in a range of 0.75 to 1.75 cm, particularly 1 to 1.5 cm, more particularly about 13.78 mm. An exterior diameter D4 of the main body 120 at the second distal end 130 may be in a range of 6 to 9 cm, particularly 6.5 to 7.5 cm, more particularly about 70 mm. An interior diameter D5 of the main body 120 at the second distal end 130 may be in a range of 5.5 to 7.5 cm, particularly 6 to 7 cm, more particularly about 66 mm. An exterior diameter D6 of the main body 120 at the first distal end 110 may be in a range of 1.25 to 2 cm, particularly 1.5 to 1.8 cm, more particularly about 16.8 mm. An interior diameter D7 of the main body 120 at the first distal end 110 may be in a range of 0.75 to 1.75 cm, particularly 1 to 1.5 cm, more particularly about 13 mm.

Flexible materials such as described above have not previously been found to be effective when used in spacer devices. This may be primarily attributed to the problem of static electricity build up that is common to flexible materials, in combination with the static resulting from movement and related friction in a collapsible configuration, such as may result in a bag or pocket having additional items therein. Static electricity in spacer devices creates a disadvantageous sticking effect for medication distributed therein, with particles sticking to the material rather than remaining airborne for inhalation by a user. However, embodiments according to the current disclosure have discovered that the use of an antistatic additive can prevent the negative drawbacks of such static electricity in the disclosed embodiments.

An antistatic additive according to the current disclosure may include a permanent antistatic additive. A permanent antistatic additive is provided in the material of the main body itself and provides an antistatic effect for at least a period of one year of use. Additives according to the current application may further allow for a collapsible spacer that is readily washable without a reduction in antistatic properties, that allows the main body to remain substantially transparent, and that is safe for use in a medical device (e.g., complying with FDA regulations and requirements). In various embodiments, an antistatic additive may comprise carbon black, graphene nanotubes, conductive mica, and/or carbon fiber. An antistatic additive may be included in the material of the main body in an amount of 0.5 to 4% by weight.

Surprisingly, the antistatic additive according to the current disclosure prevents medication from sticking to the walls of the main body and may provide an advantageously synergistic effect with the substantially conical or frustoconical shape of the main body. As discussed above, conventional thinking may lead to a belief that the substantially conical or frustoconical shape of the main body in the disclosed embodiments would cause disadvantageous impacts of a medication with the sidewalls of the spacer, reducing medication available for inhalation by a user. However, the disclosed embodiments are instead able to channel a medication to a user by a decreasing diameter or volume combined with an antistatic flexible material that reflects or otherwise does not stick to particles of medication. In this way, an amount of medication available to a user for inhalation may be maximized relative to existing spacer devices, while also adding the benefit of a collapsible configuration that is easily transported or stored.

Further, the incorporation of an antistatic additive according to the disclosed embodiments provides antistatic properties to the entire material, meaning that the drawbacks of static electricity are avoided both in an interior volume of the collapsible spacer and on an exterior surface.

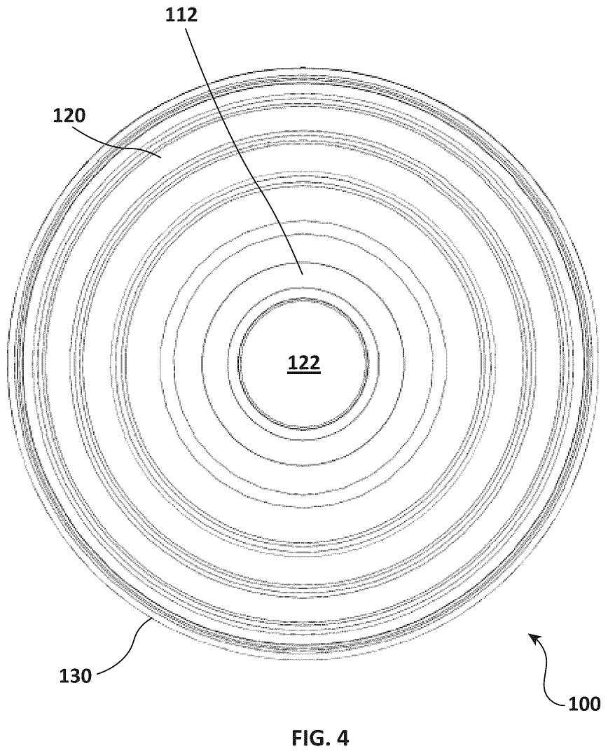

In various embodiments the main body may include ribbed elements, varying thicknesses, adhesive, snap fit connections and the like for configuring the main body to specific requirements of known inhalers, mouthpieces, valves, masks and related instruments, as would be understood by one skilled in the art from the present disclosure. The main body may be integrally formed with the outlet component, may connect to the receiver component, or otherwise be assembled in a manner appropriate to define the interior volume while remaining easy to clean. In particular, where the outlet component 112 is integrally formed or over-molded with the main body 120, as seen in the top view of FIG. 4 showing the collapsible spacer 100 in an extended configuration from the first distal end looking towards the second distal end, the collapsible spacer 100 may include only two distinct pieces such that the collapsible spacer is easily disassembled for cleaning and readily reassembled for use. The size, shape, configuration, etc., of the outlet component 112 may be selected to correspond with a corresponding mouthpiece or other device with which the spacer is used.

In some embodiments, the outlet component and/or the receiver component may comprise a material having a greater rigidity than the flexible material of the main body. Alternatively, the outlet component and/or the receiver component may comprise a flexible material like the main body but include more rigid components for defining a frame or elements for connecting with related devices. In the disclosed embodiments, the outlet component and/or the receiver component may comprise an antistatic material.

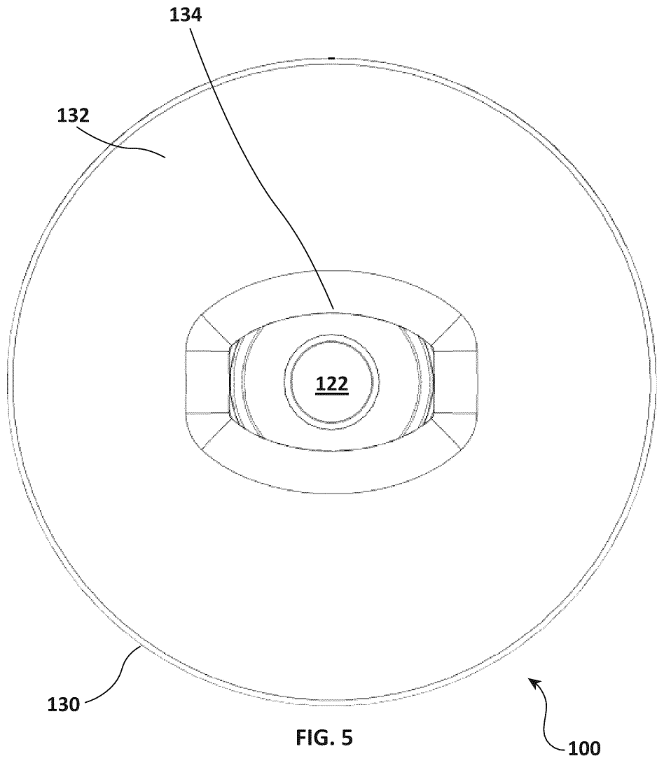

FIG. 5 illustrates a bottom view showing the collapsible spacer 100 in an extended configuration from the second distal end looking towards the first distal end, where a receiver component 132 is attached to the main body 120. As shown in the depicted embodiment, the receiver component 132 may define a recess 134 for receiving an inhaler or related device for dispensing a medication for inhalation. The size, shape, configuration, etc., of the recess may be selected to correspond with the corresponding inhaler or other device with which the spacer is used. For example, different receiver components may be provided with distinct recesses or attachment means, such that the receiver components may be exchanged according to the inhaler or device the user desires to use with the spacer. As such, different custom receiver components may be manufactured for coupling with the main body, according to varying requirements or preferences.

The outlet component may be provided with one or more ridges on an exterior surface of the outlet component. The exterior ridges may be provided to improve a connection with related devices and/or to allow a user to better grip the component for moving the collapsible spacer between a collapsed configuration and an extended configuration.

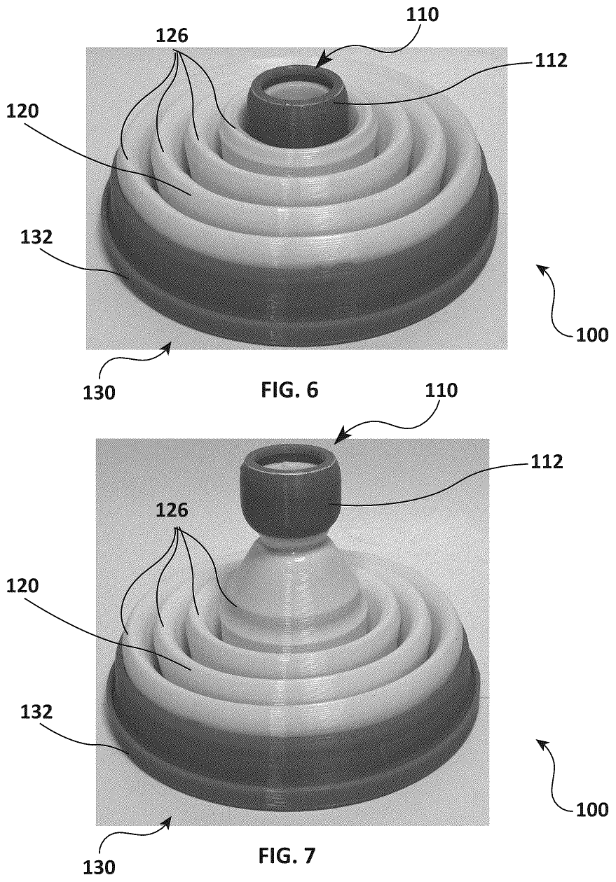

FIG. 6 illustrates a perspective view of a collapsible spacer 100 in a collapsed configuration. As shown in the depicted embodiments, a plurality of folding portions 126 defined in a main body 120 of a collapsible spacer 100 may be flexibly folded into a nested arrangement, with the folding portions 126 of the main body 120 able to collapse into a nested arrangement due to the substantially conical or frustoconical shape of the main body 120. In this manner, a more significant reduction in size may be realized without affecting the improvement provided by the collapsible spacer 100 when used in an extended configuration for administering an inhalable or respirable medication. Notably, a spacer having a substantially cylindrical shape or a substantially bicone shape cannot realize a size reduction of related degree. Rather, these other shapes would necessitate additional length, not to mention complexity, for collapsing according to the embodiments of the current disclosure. As such, the disclosed embodiments provide improvements relative to known devices, while also being manufacturable at lower cost due to the simple design of the collapsible spacer.

In the collapsed configuration of FIG. 6, the main body 120 may define a plane parallel to the receiver component 132, such as at a terminal extension of the folding portions 126. A length defined from the second distal end 130 of the collapsible spacer 100 to the plane defined by the main body 120 may be in a range of 0.75 to 2.5 cm, particularly 1 to 2 cm. A length defined from the second distal end 130 of the collapsible spacer 100 to the first distal end 110 of the collapsible spacer 100 at the outlet component 112 may be in a range of 1 to 3.5 cm, particularly 1.5 to 2.5 cm, more particularly about 2 cm.

For extending the collapsible spacer 100 from a collapsed configuration according to FIG. 6 to an extended configuration, a user may grip the outlet component 112 and the receiver component 132 and pull the components in opposite directions in order to unfold the plurality of folding portions 126 of the main body 120. Advantageously, the nested arrangement of the main body 120 in a collapsed configuration allows that a user may alternatively insert an inhaler or related device, or a finger or the like, into a recess in the receiver component to push the outlet component 112 out of the nested or collapsed configuration. In this manner, a user may easily grasp and extend the outlet component 112 away from the receiver component 132, even if it otherwise may not be possible to grip the outlet component 112 from the completely collapsed configuration unaided. This increased convenience realized by the arrangement of the disclosed embodiments further allows for a reduction in size of the collapsible spacer 100 in a collapsed configuration, meaning greater portability without sacrificing convenience or functional effectiveness.

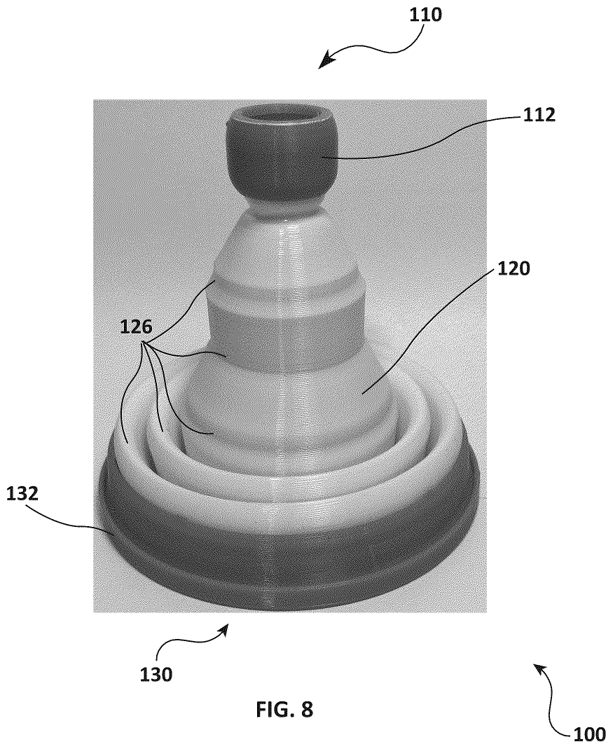

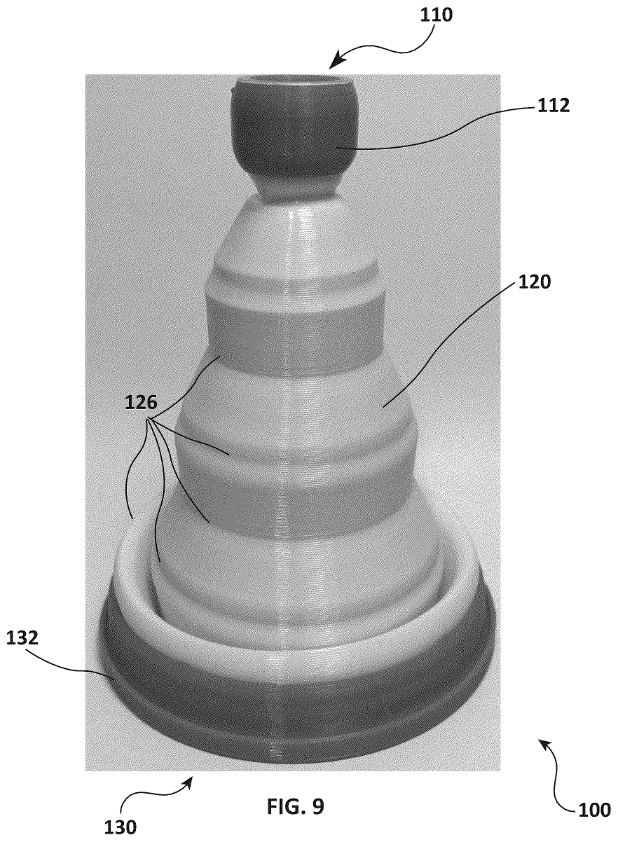

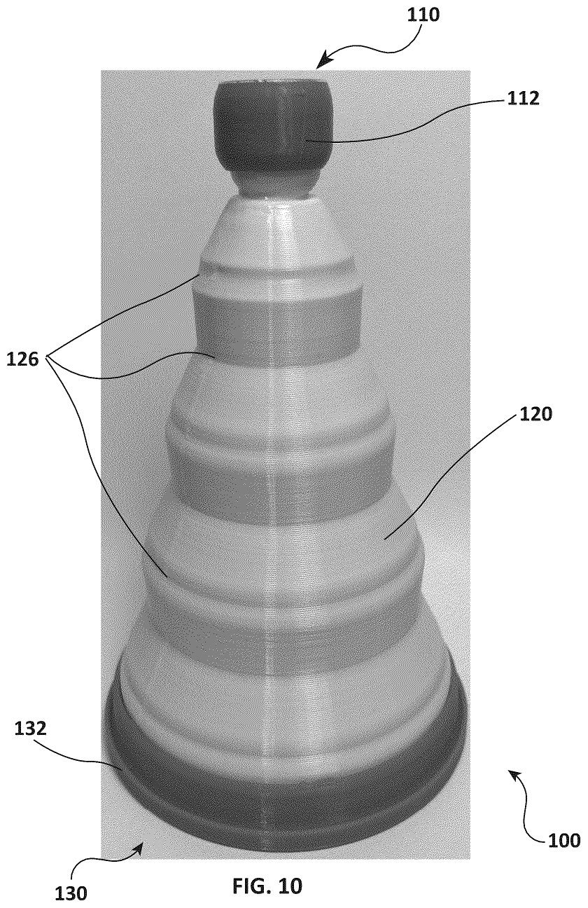

Beginning with the collapsed configuration of FIG. 6, FIGS. 7-10 illustrate a stepwise extension of an embodiment of a collapsible spacer 100 from the collapsed configuration to an extended configuration according to FIG. 10. In each step, a further folding portion 126 of the main body 120 is unfolded or extended. Notably, the collapsible spacer 100 may be extended in a different manner, e.g., in a different order of folding portions 126, and the illustrations are provided only for ease of understanding the related structure and function of the disclosed embodiments.

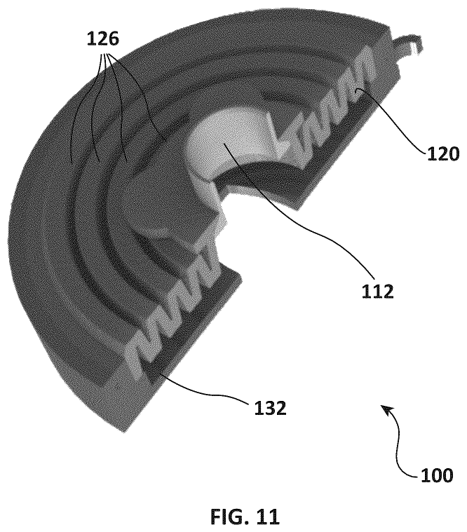

FIG. 11 further illustrates a possible cross-section of a collapsible spacer 100 in a collapsed configuration. As depicted, the nesting of the main body 120 advantageously realizes a significant reduction in size, while providing an improved convenience in use and an increased effectiveness in medication delivery. In particular, the nested configuration of the disclosed embodiments minimizes an interior volume of the collapsible spacer in a nested configuration, such that the collapsible spacer is as small as possible while still being extendable to a sufficient interior volume for improving administration of medication through inhalation. The collapsible inhaler is likewise readily adaptable for use with a variety of inhalers and related devices, without the need to modify said inhalers or related devices from their conventional state.

In view of the features of the disclosed embodiments, the collapsible spacer is advantageously adaptable and/or reconfigurable for different applications, and does not require expensive or complex, specialized equipment for use or production. Instead, the embodiments of the instant disclosure can be produced and employed without substantially increasing material or labor costs, and without complex assembly or use requirements for users.

While not described in detail, the disclosed embodiments may be combined or adapted with additional conventional features and components of spacer devices. In particular, it should be noted that a collapsible spacer of the current disclosure may be provided with one or more valves, valve assemblies and/or vents for controlling a flow of air through the device, such as for preventing a user from exhaling into an interior volume of the main body of the collapsible spacer but allowing the user to inhale therethrough, such as in valved holding chambers, or to other combinations known to those skilled in the art of spacer devices. Such valves, valve assemblies and/or vents may be provided at or in the outlet component, at or in the main body, and/or at or in the receiver component, according to various possible arrangements. Further, known sensors, guides, interactive elements, covers, cases, caps, closures, antimicrobial agents, attachment means, and the like may be included in the disclosed embodiments for increasing user compliance. Each of these additional components may be provided with antistatic properties, according to the various embodiments of the disclosure.

It is to be understood from the current disclosure that the features of the illustrated embodiments may be combined or otherwise adjusted to meet the requirements or characteristics of a particular inhaler device and/or user, such as accommodating for varying sizes, etc. Accordingly, embodiments according to the current disclosure may incorporate variations in size, shape, and/or materials, as conventionally understood in view of the current disclosure or otherwise in whole or in part from one embodiment to another.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

The disclosure further relates to several embodiments as identified by the below numbered clauses.
1. A collapsible spacer (100) comprising:
a main body (120) comprising a flexible material having antistatic properties, wherein the main body (120) is configured to move between a collapsed configuration and an extended configuration, and wherein the main body (120) in the extended configuration defines an interior volume (122) for dispersion of medication;
an outlet component (112) disposed at a first distal end (110) of the collapsible spacer (120); and
a receiver component (132) disposed at a second distal end (130) of the collapsible spacer (100), wherein the receiver component (132) is configured to allow medication to enter the interior volume (122) of the main body (120).
2. The collapsible spacer (100) according to any combination of one or more of clause 1 above and clauses 3-20 below, wherein the flexible material comprises TPE.
3. The collapsible spacer (100) according to any combination of one or more of clauses 1-2 above and clauses 4-20 below, wherein the flexible material comprises an antistatic additive.
4. The collapsible spacer (100) according to any combination of one or more of clauses 1-3 above and clauses 5-20 below, wherein the antistatic additive comprises carbon black, graphene nanotubes, conductive mica, and/or carbon fiber.
5. The collapsible spacer (100) according to any combination of one or more of clauses 1-4 above and clauses 6-20 below, wherein the main body (120) forms a cone or a frustrum in the extended configuration.
6. The collapsible spacer (100) according to any combination of one or more of clauses 1-5 above and clauses 7-20 below, wherein a diameter of the main body (120) in the extended configuration decreases monotonically from the second distal end (130) to a narrowest portion (124) of the main body (120), and the diameter of the main body (120) increases from the narrowest portion (124) to the first distal end (110).
7. The collapsible spacer (100) according to any combination of one or more of clauses 1-6 above and clauses 8-20 below, wherein the main body (120) includes a plurality of folding portions (126), the folding portions (126) configured to fold from the extended configuration into the collapsed configuration.
8. The collapsible spacer (100) according to any combination of one or more of clauses 1-7 above and clauses 9-20 below, wherein the folding portions (126) are nested together in the collapsed configuration, such that the folding portions (126) are positioned at an equal height in the collapsed configuration.
9. The collapsible spacer (100) according to any combination of one or more of clauses 1-8 above and clauses 10-20 below, wherein the first distal end (110) has a diameter smaller than a diameter of the second distal end (130).
10. The collapsible spacer (100) according to any combination of one or more of clauses 1-9 above and clauses 11-20 below, wherein the outlet component (112) is nested within a plurality of folding portions (126) of the main body (120) in the collapsed configuration.
11. The collapsible spacer (100) according to any combination of one or more of clauses 1-10 above and clauses 12-20 below, wherein the outlet component (112) comprises an exterior surface having one or more ridges.
12. The collapsible spacer (100) according to any combination of one or more of clauses 1-11 above and clauses 13-20 below, wherein the outlet component (112) and the receiver component (132) have a greater rigidity than the flexible material of the main body (120).
13. The collapsible spacer (100) according to any combination of one or more of clauses 1-12 above and clauses 14-20 below, wherein the outlet component (112) is overmolded on the main body (120).
14. The collapsible spacer (100) according to any combination of one or more of clauses 1-13 above and clauses 15-20 below, wherein a diameter of the main body (120) has a percent reduction in a range of 72% to 78% from the second distal end (130) to the first distal end (110).
15. The collapsible spacer (100) according to any combination of one or more of clauses 1-14 above and clauses 16-20 below, wherein the first distal end (110) has a diameter in a range of 1.25 to 2 cm.
16. The collapsible spacer (100) according to any combination of one or more of clauses 1-15 above and clauses 17-20 below, wherein the second distal end (130) has a diameter in a range of 6.5 to 8.0 cm.
17. The collapsible spacer (100) according to any combination of one or more of clauses 1-16 above and clauses 18-20 below, wherein the main body (120) has a wall thickness in a range of 0.25 to 0.5 cm.
18. The collapsible spacer (100) according to any combination of one or more of clauses 1-17 above and clauses 19-20 below, wherein the flexible material of the main body (120) has a shore A hardness of 25 to 80, or of 35 to 65, or 50 to 60.
19. The collapsible spacer (100) according to any combination of one or more of clauses 1-18 above and clause 20 below, wherein the main body (120) includes the antistatic additive in an amount of 0.5 to 4% by weight.
20. The collapsible spacer (100) according to any combination of one or more of clauses 1-19 above, wherein the collapsible spacer (100) has a maximum length of 10 to 13 cm in the extended configuration and of 1.5 to 2 cm in the collapsed configuration.

The present invention is in no way limited to the embodiments described by way of example and represented in the clauses.

1. A collapsible spacer comprising:
a main body comprising a flexible material having antistatic properties, wherein the main body is configured to move between a collapsed configuration and an extended configuration, and wherein the main body in the extended configuration defines an interior volume for dispersion of medication;
an outlet component disposed at a first distal end of the collapsible spacer; and
a receiver component disposed at a second distal end of the collapsible spacer, wherein the receiver component is configured to allow medication to enter the interior volume of the main body.

2. The collapsible spacer of claim 1, wherein the flexible material comprises TPE.

3. The collapsible spacer of claim 2, wherein the flexible material comprises an antistatic additive.

4. The collapsible spacer of claim 3, wherein the antistatic additive comprises carbon black, graphene nanotubes, conductive mica, and/or carbon fiber.

5. The collapsible spacer of claim 1, wherein the main body forms a cone or a frustrum in the extended configuration.

6. The collapsible spacer of claim 1, wherein a diameter of the main body in the extended configuration decreases monotonically from the second distal end to a narrowest portion of the main body, and the diameter of the main body increases from the narrowest portion to the first distal end.

7. The collapsible spacer of claim 1, wherein main body includes a plurality of folding portions, the folding portions configured to fold from the extended configuration into the collapsed configuration.

8. The collapsible spacer of claim 7, wherein the folding portions are nested together in the collapsed configuration, such that the folding portions are positioned at an equal height in the collapsed configuration.

9. The collapsible spacer of claim 1, wherein the first distal end has a diameter smaller than a diameter of the second distal end.

10. The collapsible spacer of claim 1, wherein the outlet component is nested within a plurality of folding portions of the main body in the collapsed configuration.

11. The collapsible spacer of claim 10, wherein the outlet component comprises an exterior surface having one or more ridges.

12. The collapsible spacer of claim 1, wherein the outlet component and the receiver component have a greater rigidity than the flexible material of the main body.

13. The collapsible spacer of claim 1, wherein the outlet component is overmolded on the main body.

14. The collapsible spacer of claim 1, wherein a diameter of the main body has a percent reduction in a range of 72% to 78% from the second distal end to the first distal end.

15. The collapsible spacer of claim 1, wherein the first distal end has a diameter in a range of 1.25 to 2 cm.

16. The collapsible spacer of claim 1, wherein the second distal end has a diameter in a range of 6.5 to 7.75 cm.

17. The collapsible spacer of claim 1, wherein the main body has a wall thickness in a range of 0.25 to 0.5 cm.

18. The collapsible spacer of claim 1, wherein the flexible material of the main body has a shore A hardness of 25 to 80, or of 35 to 65, or 50 to 60.

19. The collapsible spacer of claim 3, wherein the main body includes the antistatic additive in an amount of 0.5 to 4% by weight.

20. The collapsible spacer of claim 1, wherein the collapsible spacer has a maximum length of 10 to 13 cm in the extended configuration and of 1.5 to 2 cm in the collapsed configuration.
